(19) United States
(12) Patent Application Publication
(10) Pub. No.: US 2022/0202722 A1
(43) Pub. Date: Jun. 30, 2022

(54) ORODISPERSIBLE TABLET

(71) Applicant: Reckitt Benckiser Health Limited, Bekshire (GB)

(72) Inventors: Steven Louis BARNETT, Humberside (GB); Calum McIntosh GORDON, Humberside (GB); Jennifer Esme HANNING, Humberside (GB); Tracy Emmanuelle KLÔH, Humberside (GB)

(73) Assignee: Reckitt Benckiser Health Limited, Berkshire (GB)

(21) Appl. No.: 17/425,981

(22) PCT Filed: Jan. 28, 2020
(86) PCT No.: PCT/GB2020/050193

§ 371 Date: Jul. 27, 2021

(30) Foreign Application Priority Data
Jan. 28, 2019 (GB) 1901137.8

(51) Int. Cl. A61K 9/20; A61K 31/192; A61K 9/00

(52) U.S. Cl. CPC A61K 9/2077; A61K 31/192; A61K 9/2054; A61K 9/0056; A61K 9/2027; A61K 9/2009; A61K 9/2018

(57) ABSTRACT

The present invention is directed to a composition in the form of an orodispersible tablet comprising a non-steroidal anti-inflammatory drug (NSAID) and a taste-masking agent wherein the NSAID is uncoated and has an average particle size of less than 100 μm. The present invention is further directed to the use of malic acid as a taste-masking agent.

The present invention relates to a tablet, in particular, an orodispersible tablet and a method of masking the taste of the actives in the orodispersible tablet.

Orodispersible tablets are compressed tablets which are pressed to a lower hardness than conventional tablets so that they will disintegrate in the mouth of the consumer. Although orodispersible tablets disintegrate in the mouth the absorption of the active substance may not be via the oral mucosa but via the stomach or small intestine.

Advantageously, therefore, orodispersible tablets can be taken without water to deliver actives to the stomach. Such a mechanism of delivery is not only convenient but can be beneficial when there may be difficulty swallowing conventional tablets such as with young children, the elderly or very infirm patients.

However, some actives must be taste-masked if they are to be included in an orodispersible tablet due to their bitter taste and/or ‘throat-catch’ which is common to many medicaments. For example, ibuprofen is a well-known medicament which produces an unpalatable burning sensation in the mouth and throat after ingestion.

In the art, several methods of taste-masking or mitigating ‘throat-catch’ are known. U.S. Pat. No. 8,496,969 describes the use of a coated particle of ibuprofen to form a taste-masked tablet. The taste-masking agent comprises a combination of cellulose acetate, hypromellose phthalate and polysorbate-80 and is provided as a solution wherein the solvent is 90% acetone and 10% water. Prior to coating the ibuprofen is granulated to a size of over 200 μm. The use of a coating can impact on the release of the ibuprofen. U.S. Pat. No. 5,405,617 is directed to the use of an aliphatic or fatty acid ester to coat an active pharmaceutical ingredient. The aliphatic acid or ester is melted prior to coating the active ingredient. WO 2013/143688 describes a process wherein an active pharmaceutical ingredient is taste-masked by melt extrusion with a suitable excipient. The excipient is selected from the group consisting of fatty acids, fatty acid salts or esters, glyceride esters, waxes, polyvinyl caprolactam-polyvinyl acetate polyethylene glycol graft copolymer or combinations thereof.

However, these drugs can also be difficult to taste-mask using standard dosage forms due to the fact they will quickly dissolve in saliva and reach the taste buds. In particular, drug substances which have a small particle size will have an increased surface area and as a result will contact more taste receptors thereby making effective taste-masking more challenging. In contrast, drug substances with a larger particle size will have a reduced surface area and as a result will contact fewer taste receptors. Accordingly, compositions which contain drug substances that require taste-masking will generally use drugs having a higher particle size to reduce the necessity for taste-masking as a result of their lower surface area. Lower particle size grades of NSAIDs, such as ibuprofen, are known to be particularly difficult to taste-mask effectively and as a result are not used in current orodispersible products.

However, it is highly desirable for orodispersible compositions to exhibit a mouth-feel that is acceptable to a patient. Orodispersible compositions which comprise a drug substance having a higher particle size generally have an unpleasant or undesirable ‘gritty’ feel when they dissolve in the mouth.

In addition, orodispersible tablets can often have a high friability and low hardness to ensure that they disintegrate as required when placed in the oral cavity. However, manufacturing orodispersible tablets which have the required disintegration profile and the necessary hardness and friability such that they are a viable commercial product can be difficult.

It would, therefore, be desirable to have an orodispersible tablet which comprises an uncoated active pharmaceutical and has a ‘smooth’ mouth feel but does not exhibit an unpleasant taste.

Taste-masking such of orodispersible compositions which comprise an uncoated active pharmaceutical and have a ‘smooth’ mouth feel can require a large amount of taste-masking agent which can make the size of the tablet larger than is acceptable to a consumer.

It would, therefore, also be desirable to have an orodispersible tablet which comprises an uncoated active pharmaceutical and has a ‘smooth’ mouth feel but does not require the inclusion of a large amount of a taste-masking agent.

According to an aspect the present invention there is provided a composition in the form of an orodispersible tablet comprising a non-steroidal anti-inflammatory drug (NSAID) and a taste-masking agent wherein the NSAID is uncoated and has an average particle size of less than 100 μm.

The average particle size of the uncoated NSAID can be less than 90 μm. The average particle size of the uncoated NSAID can be less than 80 μm. The average particle size of the uncoated NSAID can be less than 70 μm. The average particle size of the uncoated NSAID can be less than 60 μm. The average particle size of the uncoated NSAID can be greater than 10 μm. The average particle size of the uncoated NSAID can be greater than 20 μm. The average particle size of the uncoated NSAID can be greater than 30 μm. The average particle size of the uncoated NSAID can be greater than 40 μm.

The average particle size of the uncoated NSAID can be from 10 μm-80 μm. The average particle size of the uncoated NSAID can preferably be from 30 μm-70 μm. The average particle size of the uncoated NSAID can more preferably be from 40-60 μm. A most preferred average particle size is 50 μm.

Preferably, the taste-masking agent does not comprise a cyclodextrin.

Typically, the dosage amount for a pharmaceutical active in the treatment of pain, inflammation or fever is in the range 25-600 mg, more preferably 100-400 mg depending upon the suitable dose level of the active pharmaceutical agent. A preferred dosage amount is 200 mg. An especially preferred dosage amount for the composition of the present invention is 200 mg ibuprofen. Where derivatives are employed, normally the precise unit dosages are chosen to give the equivalent NSAID doses given above.

The most preferred active agents are those used in the treatment of pain, particularly, ibuprofen, flurbiprofen, ketoprofen, aspirin, paracetamol, codeine, naproxen, indomethacin, diclofenac and meloxicam. Especially preferred is ibuprofen and its pharmaceutically acceptable salts or hydrates.

The taste-masking agent can be selected from the group consisting of ascorbic acid, monosodium citrate, fumaric acid, malic acid, and combinations thereof. A preferred taste-masking agent is malic acid.

The taste masking agent can be present at a level of up to 15%. The amount of taste-masking agent can be at least 0.1%. The amount of taste-masking agent can be at least 0.5%. The amount of taste-masking agent can be at least 1%. The amount of taste-masking agent can be at least 5%. The amount of taste-masking agent can be at least 7%.

The amount of taste masking agent can be up to 10%. The amount of taste masking agent can be up to 9%. The amount of taste masking agent can be up to 2%. The amount of taste masking agent can be up to 1%.

When the taste-masking agent is selected to be malic acid, the amount present can be 0.1-15%. Preferably, the amount can be 5-10%. More preferably the amount can be 7-9%.

The ratio by weight of the non-steroidal anti-inflammatory drug (NSAID) and the taste-masking agent in the composition can be from 1:1-8:1. A preferred ratio is 3:1-5:1. A most preferred ratio is 4:1 by weight.

For a composition of the present invention, i.e. an orodispersible tablet, the active ingredient will generally be combined with various excipients. In particular, the composition will generally comprise one or more lubricants, glidants, fillers, binders, disintegrants, diluents or bulking agents.

Typically, the composition can include additional flavouring agents. The flavouring agent can be any suitable flavour, such as orange, cherry, cola, grapefruit, raspberry, lemon, mulberry, apricot or a cooling flavour.

The flavouring agent can be present at a level of up to 5%. The flavouring agent can be present at a level of 0.1%-3%.

The composition can further include a sweetener. The sweetener can be selected from the group consisting of mannitol, sorbitol, isomalt, sodium saccharin, acesulfame potassium, sucralose, aspartame, monoammonium glycyrrhizinate, neohesperidin dihydrochalcone, thaumatin, neotame, cyclamates, and mixtures thereof. A preferred sweetener is sucralose.

Preferred materials for the filler include celluloses, polysaccharides and derivatives thereof, sugar alcohols and saccharides.

Polysaccharides which may be used include starch, e.g. maize starch, cellulose, e.g. powdered cellulose and microcrystalline cellulose, water-insoluble modified starches, e.g. sodium carboxymethyl starch, water-insoluble cellulose derivatives, e.g. croscarmellose sodium (cross-linked sodium carboxymethyl cellulose), cross-linked polyvinylpyrrolidone, maltodextrin and alginic acid.

Another preferred form of filler is a saccharide. Suitable saccharides include, for example, sucrose, lactose and dextrose.

Another preferred form of filler is a sugar alcohol. Suitable sugar alcohols include, for example, sorbitol, mannitol and, xylitol. Mannitol is especially preferred.

Typically, the filler can be present at a level of 20-70% by weight of the composition. More typically, the filler can be present at a level of 40-50% by weight of the composition. Most typically the filler can be present at a level of 42-47% by weight of the composition.

Particularly preferred binders are dicalcium phosphate, microcrystalline cellulose, e.g. the products sold as Avicel PH-101 and Avicel PH-102 (Avicel is a Trade Mark) and lactose. A preferred binder is microcrystalline cellulose. Typically, the binder can be present at a level of 2-25% by weight of the composition. More typically, the binder can be present at a level of 5-15% by weight of the composition. Most typically the binder can be present at a level of 8-12% by weight of the composition.

The disintegrant has the effect of assisting the orodispersible tablet to disintegrate in the buccal cavity. Apart from croscarmellose sodium, examples of disintegrants include one or more of wheat starch, maize starch, potato starch, sodium starch glycolate, low-substituted hydroxypropyl cellulose, alginic acid, cross-linked polyvinylpyrrolidone (crospovidone) and magnesium aluminium silicate. Preferred disintegrants are those which swell on the action of water thus causing the ingredients in the orodispersible tablet to be pushed apart and out into the aqueous disintegration medium. A preferred disintegrant is cross-linked polyvinylpyrrolidone (crospovidone). The disintegrant is present at an effective disintegrating amount, for example up to 20% by weight of the composition, more preferably 1-10% w/w, further preferably 2-8% w/w and most preferably 3-6% by weight of the composition.

A particularly preferred composition includes a blend of a cellulosic binder, a sugar alcohol filler and a disintegrant. The preferred cellulosic binder is microcrystalline cellulose, the preferred sugar alcohol is mannitol, and the preferred disintegrant is cross-linked polyvinylpyrrolidone (crospovidone).

Typically, the composition comprises the cellulosic binder, the sugar alcohol filler and the disintegrant in the ratio of 0.01-10 parts by weight of cellulosic binder: 0.01-10 parts by weight of sugar alcohol filler:1 part by weight of disintegrant. More preferably, the composition contains 2-4 parts by weight of cellulosic binder per part by weight of disintegrant, and 8-12 parts by weight of sugar alcohol filler per part by weight of disintegrant. Most preferably, the composition contains cellulosic binder, sugar alcohol filler and disintegrant in a weight ratio of 2-3:11-12:1 parts by weight. Preferably, the composition contains cellulosic binder, sugar alcohol filler and disintegrant in a weight ratio of 2.5:11.5:1. Alternatively, the composition can contain cellulosic binder, sugar alcohol filler and disintegrant in a weight ratio of 2.5:11.4:1. Alternatively, the composition contains cellulosic binder, sugar alcohol filler and disintegrant in a weight ratio of 2.5:11.05:1.

The binder, filler and/or disintegrant are preferably incorporated into the composition in finely divided (powder) form.

The binder and filler preferably together constitute in excess of 40% w/w of the composition, more preferably in excess of 45% w/w, and most preferably in excess of 50% w/w. The binder and filler preferably together constitute less than 65% w/w of the composition, more preferably less than 70% w/w, and most preferably less than 75% w/w. The binder and filler preferably together constitute between 40 and 75% w/w of the composition, more preferably between 45 and 70% w/w, and most preferably between 50 and 65% w/w. Preferably, the binder and filler together constitute between 50 and 56% w/w of the composition. Alternatively, the binder and filler together can constitute between 60-65% w/w.

The lubricant may be, for example, stearic acid, a stearate, a fumarate, or a polyethylene glycol of molecular weight of 4,000 or more. The preferred lubricant is a metallic fumarate, particularly sodium stearyl fumarate, which lubricant may be present in the composition at low levels of up to 5%, typically than 1% or 3% by weight either alone or in combination. The lubricant can be present at a level of 1.25%.

The glidant can be selected from the group consisting of silica, talc or starch. The glidant is typically present at a level of up to 5%. The glidant can be present at a level of 0.1%-4%. The glidant can be present at a level of 0.5%-2%. The glidant can be present at a level of 0.75%-1.5%. The glidant can be present at a level of 1%.

For the avoidance of doubt, in the context of the present application a glidant is a component which ensures the powder which forms the orodispersible tablet flows easily into the dies of the tablet press, and a lubricant is defined as being a component which aids ejection of the orodispersible tablet from the dies of the press and prevents the orodispersible tablet from sticking to the punches.

The composition can have a friability of up to 2% w/w and a hardness of between 40N and 100N. The composition can have a friability of less than 1% w/w and a hardness of greater than 50N. The composition can have a friability of less than 1% w/w and a hardness of greater than 60N. The composition can have a friability of less than 1% w/w and a hardness of greater than 70N. The composition can have a friability of less than 1% w/w and a hardness of greater than 80N. The composition can have a friability of less than 0.5% w/w and a hardness of greater than 50N. The composition can have a friability of less than 0.5% w/w and a hardness of greater than 60N. The composition can have a friability of less than 0.5% w/w and a hardness of greater than 70N. The composition can have a friability of less than 0.5% w/w and a hardness of greater than 80N.

Preferably, the composition can have a friability of less than 1% w/w and a hardness of greater than 80N. More preferably, the composition can have a friability of less than 0.5% w/w and a hardness of greater than 80N.

The composition can comprise an uncoated non-steroidal anti-inflammatory drug (NSAID), a taste-masking agent and a disintegrant wherein the uncoated NSAID has an average particle size of less than 100 μm, the taste-masking agent is selected to be malic acid which can be present in an amount of 0.1-15% by weight of the composition, and the disintegrant is selected to be cross-linked polyvinylpyrrolidone (crospovidone) which can be present in an amount of 1-10% by weight of the composition.

Typically, the amount of malic acid can be 5-10% by weight of the composition. More typically the amount of malic acid can be 7-9% by weight of the composition.

Typically, the amount of crospovidone can be 2-8% by weight of the composition and more typically 3-6% by weight of the composition.

The composition can comprise an uncoated non-steroidal anti-inflammatory drug (NSAID), a taste-masking agent and a disintegrant wherein the uncoated NSAID has an average particle size of less than 100 μm, the taste-masking agent is selected to be malic acid which can be present in an amount of 0.1-15% by weight of the composition, wherein the ratio of the uncoated non-steroidal anti-inflammatory drug (NSAID) to taste-masking agent is 4:1 by weight. The average particle size of the uncoated NSAID can be from 40-60 μm. A preferred average particle size of the NSAID is 50 μm. Preferably, the uncoated non-steroidal anti-inflammatory drug (NSAID) is ibuprofen. Typically, the amount of malic acid can be 5-10% by weight of the composition. More typically the amount of malic acid can be 7-9% by weight of the composition.

The composition can comprise an uncoated non-steroidal anti-inflammatory drug (NSAID), a taste-masking agent and a disintegrant wherein the uncoated NSAID has an average particle size of less than 100 μm, the taste-masking agent is selected to be malic acid which can be present in an amount of 0.1-15% by weight of the composition, wherein the ratio of the uncoated non-steroidal anti-inflammatory drug (NSAID) to taste-masking agent is 4:1 by weight and the disintegrant is selected to be cross-linked polyvinylpyrrolidone (crospovidone) which can be present in an amount of 1-10% by weight of the composition. The average particle size of the uncoated NSAID can be from 40-60 μm. A preferred average particle size of the NSAID is 50 μm. Preferably, the uncoated non-steroidal anti-inflammatory drug (NSAID) is ibuprofen. Typically, the amount of malic acid can be 5-10% by weight of the composition. More typically the amount of malic acid can be 7-9% by weight of the composition. Typically, the amount of crospovidone can be 2-8% by weight of the composition and more typically 3-6% by weight of the composition.

The composition can comprise uncoated non-steroidal anti-inflammatory drug (NSAID), a taste-masking agent and a disintegrant wherein the uncoated NSAID has an average particle size of 50 μm, the taste-masking agent is selected to be malic acid which can be present in an amount of 7-9% by weight of the composition, wherein the ratio of the uncoated non-steroidal anti-inflammatory drug (NSAID) to taste-masking agent is 4:1 by weight and the disintegrant is selected to be cross-linked polyvinylpyrrolidone (crospovidone) which can be present in an amount of 3-6% by weight of the composition.

The composition can further comprise 0.1%-2% w/w of a colour.

The composition can comprise uncoated ibuprofen, a taste-masking agent and a disintegrant wherein the uncoated ibuprofen has an average particle size of less than 100 μm and wherein the weight ratio of ibuprofen:taste-masking agent:disintegrant is from about 10:5:1 to about 5:1:1. Typically, the composition can comprise uncoated ibuprofen, a taste-masking agent and a disintegrant wherein the weight ratio of ibuprofen:taste-masking agent:disintegrant is from about 8:2:1 to about 7:1:1.

The composition can comprise uncoated ibuprofen, a taste-masking agent and a disintegrant wherein the uncoated ibuprofen has an average particle size of less than 100 μm, the taste-masking agent is selected to be malic acid which can be present in an amount of 0.1-15% by weight of the composition, wherein the ratio of the uncoated ibuprofen to taste-masking agent is 4:1 by weight and the disintegrant is selected to be cross-linked polyvinylpyrrolidone (crospovidone) which can be present in an amount of 1-10% by weight of the composition and wherein the weight ratio of ibuprofen:malic acid:cross-linked polyvinylpyrrolidone is from about 10:5:1 to about 5:1:1. The average particle size of the uncoated ibuprofen can be from 40-60 μm. A preferred average particle size of the NSAID is 50 μm. Typically, the weight ratio of the uncoated ibuprofen, malic acid, cross-linked polyvinyl pyrrolidone is from about 8:2:1 to about 7:1:1.

The composition can comprise uncoated ibuprofen, a taste-masking agent and a disintegrant wherein the uncoated ibuprofen has an average particle size of 50 μm, the taste-masking agent is selected to be malic acid which can be present in an amount of 7-9% by weight of the composition, wherein the ratio of the uncoated ibuprofen to taste-masking agent is 4:1 by weight and the disintegrant is selected to be cross-linked polyvinylpyrrolidone (crospovidone) which can be present in an amount of 3-6% by weight of the composition and wherein the weight ratio of ibuprofen:taste-masking agent:disintegrant is from about 8:2:1 to about 7:1:1.

The composition can comprise an uncoated non-steroidal anti-inflammatory drug (NSAID), a taste-masking agent, a binder, a disintegrant and a filler wherein the uncoated NSAID has an average particle size of less than 100 μm, the taste-masking agent can be present in an amount of 0.1-15% by weight of the composition, wherein the ratio of the NSAID to taste-masking agent is 4:1 by weight and wherein the composition contains 2-4 parts by weight of binder per part by weight of disintegrant, and 8-12 parts by weight of filler per part by weight of disintegrant. The average particle size of the uncoated NSAID can be from 40-60 μm. A preferred average particle size of the NSAID is 50 μm. Preferably, the uncoated non-steroidal anti-inflammatory drug (NSAID) is ibuprofen. Typically, the amount of taste-masking agent can be 5-10% by weight of the composition. More typically the amount of taste-masking agent can be 7-9% by weight of the composition.

The composition can comprise an uncoated non-steroidal anti-inflammatory drug (NSAID), a taste-masking agent, a cellulosic binder, a disintegrant and a sugar alcohol filler wherein the uncoated NSAID has an average particle size of less than 100 μm, the taste-masking agent is selected to be malic acid which can be present in an amount of 0.1-15% by weight of the composition, wherein the ratio of the NSAID to taste-masking agent is 4:1 by weight and wherein the composition contains 2-4 parts by weight of cellulosic binder per part by weight of disintegrant, and 8-12 parts by weight of sugar alcohol filler per part by weight of disintegrant. The average particle size of the uncoated NSAID can be from 40-60 μm. A preferred average particle size of the NSAID is 50 μm. Preferably, the uncoated non-steroidal anti-inflammatory drug (NSAID) is ibuprofen. Typically, the amount of malic acid can be 5-10% by weight of the composition. More typically the amount of malic acid can be 7-9% by weight of the composition.

The composition can comprise an uncoated non-steroidal anti-inflammatory drug (NSAID), a taste-masking agent and a disintegrant wherein the uncoated NSAID has an average particle size of less than 100 μm, the taste-masking agent is selected to be malic acid which can be present in an amount of 0.1-15% by weight of the composition, wherein the ratio of the uncoated non-steroidal anti-inflammatory drug (NSAID) to taste-masking agent is 4:1 by weight and the disintegrant is selected to be cross-linked polyvinylpyrrolidone (crospovidone) which can be present in an amount of 1-10% by weight of the composition. The average particle size of the uncoated NSAID can be from 40-60 μm. A preferred average particle size of the NSAID is 50 μm. Preferably, the uncoated non-steroidal anti-inflammatory drug (NSAID) is ibuprofen. Typically, the amount of malic acid can be 5-10% by weight of the composition. More typically the amount of malic acid can be 7-9% by weight of the composition. Typically, the amount of crospovidone can be 2-8% by weight of the composition and more typically 3-6% by weight of the composition.

The composition can be in the form of an orodispersible tablet comprising uncoated ibuprofen, a taste-masking agent, a cellulosic binder, a disintegrant and a sugar alcohol filler wherein the ibuprofen has an average particle size of 50 μm, the taste-masking agent is selected to be malic acid and is present in an amount of 0.1-15% by weight of the composition, wherein the ratio of the uncoated ibuprofen to taste-masking agent is 4:1 by weight, the cellulosic binder is selected to be microcrystalline cellulose and is present in an amount of 2-25% by weight of the composition, the disintegrant is selected to be cross-linked polyvinylpyrrolidone (crospovidone) and is present in an amount of 1-10% by weight of the composition, the filler is selected to be mannitol and is present in an amount of 20-70% by weight of the composition, wherein the composition contains 2-4 parts by weight of cellulosic binder per part by weight of disintegrant, and 8-12 parts by weight of sugar alcohol filler per part by weight of disintegrant, and wherein the weight ratio of ibuprofen:malic acid:cross-linked polyvinylpyrrolidone is from about 10:5:1 to about 5:1:1.

Typically, the amount of malic acid can be 5-10% by weight of the composition. More typically the amount of malic acid can be 7-9% by weight of the composition. Typically, the microcrystalline cellulose can be present at a level of 5-15% by weight of the composition. More typically the microcrystalline cellulose can be present at a level of 8-12% by weight of the composition. Typically, the amount of crospovidone can be 2-8% by weight of the composition. More typically the amount of crospovidone can be 3-6% by weight of the composition. Typically, the mannitol can be present at a level of 40-50% by weight of the composition. More typically, the mannitol can be present at a level of 42-47% by weight of the composition. The composition can contain cellulosic binder, sugar alcohol filler and disintegrant in a weight ratio of 2-3:11-12:1 parts by weight. Typically, the weight ratio of the ibuprofen:malic acid:cross-linked polyvinyl pyrrolidone is from about 8:2:1 to about 7:1:1.

Preferably, the composition can be in the form of an orodispersible tablet comprising uncoated ibuprofen, a taste-masking agent, a cellulosic binder, a disintegrant and a sugar alcohol filler wherein the ibuprofen has an average particle size of 50 μm, the taste-masking agent is selected to be malic acid and is present in an amount of 7-9% by weight of the composition, wherein the ratio of the NSAID to taste-masking agent is 4:1 by weight, the cellulosic binder is selected to be microcrystalline cellulose and is present in an amount of 8-12% by weight of the composition, the disintegrant is selected to be cross-linked polyvinylpyrrolidone (crospovidone) and is present in an amount of 3-6% by weight of the composition, the filler is selected to be mannitol and is present in an amount of 42-47% by weight of the composition, cellulosic binder, sugar alcohol filler and disintegrant in a weight ratio of 2-3:11-12:1 parts by weight, and wherein the weight ratio of ibuprofen:malic acid:cross-linked polyvinylpyrrolidone is from about 8:2:1 to about 7:1:1.

The composition can comprise:
(a) 20-35% of an uncoated NSAID having an average particle size of less than 100 μm or a pharmaceutically acceptable salt thereof;
(b) 20-70% of a filler;
(c) 0.5-5% of a lubricant;
(d) 5-10% of a taste-masking agent; and
(e) 1-10% of a disintegrant.

The average particle size of the uncoated NSAID can be from 40-60 μm. A preferred average particle size of the uncoated NSAID is 50 μm.

The typically further comprises one or more additional excipients selected from:
(a) 0.1-5% of a glidant; and
(b) 2-25% of a binder.

The composition can comprise:
(a) 25-30% of an uncoated NSAID having an average particle size of less than 100 μm or a pharmaceutically acceptable salt thereof;
(b) 40-50% of a filler;
(c) 0.5-5% of a lubricant;
(d) 5-10% of a taste-masking agent; and
(e) 1-10% of a disintegrant.

The average particle size of the uncoated NSAID can be from 40-60 μm. A preferred average particle size of the uncoated NSAID is 50 μm.

The composition typically further comprises one or more additional excipients selected from:
(a) 0.1-5% of a glidant; and
(b) 2-25% of a binder.

Alternatively, the composition can comprise:
(a) 20-35% of an uncoated NSAID having an average particle size of less than 100 μm or a pharmaceutically acceptable salt thereof;
(b) 0.1-5% of a glidant;
(c) 20-70% of a filler;
(d) 0.5-5% of a lubricant;
(e) 2-25% of a binder;
(f) 5-10% of a taste-masking agent; and
(g) 1-10% of a disintegrant.

The average particle size of the uncoated NSAID can be from 40-60 μm. A preferred average particle size of the uncoated NSAID is 50 μm.

The composition can comprise:
(a) 25-30% of an uncoated NSAID having an average particle size of less than 100 μm or a pharmaceutically acceptable salt thereof;
(b) 0.1-5% of a glidant;
(c) 40-50% of a filler;
(d) 0.5-5% of a lubricant;
(e) 5-15% of a binder;
(f) 5-10% of a taste-masking agent; and
(g) 1-10% of a disintegrant.

The average particle size of the uncoated NSAID can be from 40-60 μm. A preferred average particle size of the uncoated NSAID is 50 μm.

The composition can comprise:
(a) 28-30% of an uncoated NSAID having an average particle size of less than 100 μm or a pharmaceutically acceptable salt thereof;
(b) 0.5-1.5% of a glidant;
(c) 42-47% of a filler;
(d) 0.75-1.5% of a lubricant;
(e) 8-12% of a binder;
(f) 6-8% of a taste-masking agent; and
(g) 3-5% of a disintegrant.

The average particle size of the uncoated NSAID can be from 40-60 μm. A preferred average particle size of the uncoated NSAID is 50 μm.

The composition can comprise:
(a) 25-30% of uncoated ibuprofen having an average particle size of less than 100 μm or a pharmaceutically acceptable salt thereof;
(b) 0.1-5% of colloidal silica;
(c) 40-50% of mannitol;
(d) 0.5-5% of sodium stearyl fumarate;
(e) 5-15% of microcrystalline cellulose;
(f) 5-10% of malic acid; and
(g) 1-10% of crospovidone.

The average particle size of the uncoated ibuprofen can be from 40-60 μm. A preferred average particle size of the uncoated ibuprofen is 50 μm.

The composition can most preferably comprise:
(a) 28-30% of uncoated ibuprofen having an average particle size of less than 100 μm or a pharmaceutically acceptable salt thereof;
(b) 0.5-1.5% of a colloidal silica;
(c) 42-47% of a mannitol;
(d) 0.75-1.5% of a sodium stearyl fumarate;
(e) 8-12% of a microcrystalline cellulose;
(f) 6-8% of a malic acid; and
(g) 3-5% of a crospovidone.

The average particle size of the uncoated ibuprofen can be from 40-60 μm. A preferred average particle size of the uncoated ibuprofen is 50 μm.

The composition can consist essentially of an uncoated non-steroidal anti-inflammatory drug (NSAID), a taste-masking agent and a disintegrant wherein the uncoated NSAID has an average particle size of less than 100 μm, the taste-masking agent is selected to be malic acid which can be present in an amount of 0.1-15% by weight of the composition, wherein the ratio of the uncoated non-steroidal anti-inflammatory drug (NSAID) to taste-masking agent is 4:1 by weight. The average particle size of the uncoated NSAID can be from 40-60 μm. A preferred average particle size of the NSAID is 50 μm. Typically, the uncoated NSAID is selected to be ibuprofen. Typically, the amount of malic acid can be 5-10% by weight of the composition. More typically the amount of malic acid can be 7-9% by weight of the composition. Typically, the disintegrant is selected to be crospovidone which is present in an amount of 2-8% by weight of the composition. More typically, the crospovidone is present in an amount of 3-6% by weight of the composition. Typically, the weight ratio of the ibuprofen:malic acid:cross-linked polyvinyl pyrrolidone is from about 8:2:1 to about 7:1:1.

More typically, the binder can be present at a level of 40-50% by weight of the composition. Most typically the binder can be present at a level of 42-47% by weight of the composition.

The composition can contain cellulosic binder, sugar alcohol filler and disintegrant in a weight ratio of 2-3:11-12:1 parts by weight.

The composition can consist essentially of an uncoated non-steroidal anti-inflammatory drug (NSAID), a taste-masking agent and a disintegrant wherein the uncoated NSAID has an average particle size of less than 100 μm, the taste-masking agent is selected to be malic acid which can be present in an amount of 0.1-15% by weight of the composition, wherein the ratio of the uncoated non-steroidal anti-inflammatory drug (NSAID) to taste-masking agent is 4:1 by weight and the disintegrant is selected to be cross-linked polyvinylpyrrolidone (crospovidone) which can be present in an amount of 1-10% by weight of the composition. The average particle size of the uncoated NSAID can be from 40-60 μm. A preferred average particle size of the NSAID is 50 μm. Typically, the uncoated NSAID is selected to be ibuprofen.

The composition can consist essentially of an uncoated non-steroidal anti-inflammatory drug (NSAID), a taste-masking agent and a disintegrant wherein the uncoated NSAID has an average particle size of less than 100 μm, the taste-masking agent is selected to be malic acid which can be present in an amount of 0.1-15% by weight of the composition, wherein the ratio of the uncoated non-steroidal anti-inflammatory drug (NSAID) to taste-masking agent is 4:1 by weight and the disintegrant is selected to be cross-linked polyvinylpyrrolidone (crospovidone) which can be present in an amount of 1-10% by weight of the composition. The average particle size of the uncoated NSAID can be from 40-60 μm. A preferred average particle size of the NSAID is 50 μm. Typically, the uncoated NSAID is selected to be ibuprofen.

Preferably, the composition can have a friability of less than 1% w/w and a hardness of greater than 80N. More preferably, the composition can have a friability of less than 0.5% w/w and a hardness of greater than 80N.

The composition can be in the form of an orodispersible tablet consisting essentially of uncoated ibuprofen, a taste-masking agent, a cellulosic binder, a disintegrant and a sugar alcohol filler wherein the ibuprofen has an average particle size of 50 μm, the taste-masking agent is selected to be malic acid and is present in an amount of 7-9% by weight of the composition, wherein the ratio of the NSAID to taste-masking agent is 4:1 by weight, the cellulosic binder is selected to be microcrystalline cellulose and is present in an amount of 8-12% by weight of the composition, the disintegrant is selected to be cross-linked polyvinylpyrrolidone (crospovidone) and is present in an amount of 3-6% by weight of the composition, the filler is selected to be mannitol and is present in an amount of 42-47% by weight of the composition, cellulosic binder, sugar alcohol filler and disintegrant in a weight ratio of 2-3:11-12:1 parts by weight, and wherein the weight ratio of ibuprofen:malic acid:cross-linked polyvinylpyrrolidone is from about 8:2:1 to about 7:1:1.

The composition can be in the form of an orodispersible tablet consisting essentially of uncoated ibuprofen, a taste-masking agent, a cellulosic binder, a disintegrant and a sugar alcohol filler wherein the ibuprofen has an average particle size of 50 μm, the taste-masking agent is selected to be malic acid and is present in an amount of 7-9% by weight of the composition, wherein the ratio of the NSAID to taste-masking agent is 4:1 by weight, the cellulosic binder is selected to be microcrystalline cellulose and is present in an amount of 8-12% by weight of the composition, the disintegrant is selected to be cross-linked polyvinylpyrrolidone (crospovidone) and is present in an amount of 3-6% by weight of the composition, the filler is selected to be mannitol and is present in an amount of 42-47% by weight of the composition, cellulosic binder, sugar alcohol filler and disintegrant in a weight ratio of 2-3:11-12:1 parts by weight, and wherein the weight ratio of ibuprofen:malic acid:cross-linked polyvinylpyrrolidone is from about 8:2:1 to about 7:1:1 and wherein the composition has a friability of less than 0.5% w/w and a hardness of greater than 80N.

The composition can consist essentially of:
(a) 20-35% of an uncoated NSAID having an average particle size of less than 100 μm or a pharmaceutically acceptable salt thereof;
(b) 5-10% of a taste-masking agent;
(c) 1-10% of a disintegrant; and
(d) 2-25% of a binder.

The average particle size of the uncoated NSAID can be from 40-60 μm. A preferred average particle size of the uncoated NSAID is 50 μm. The composition can further include one or more of 20-70% filler, 0.5-5% lubricant and 0.1-5% glidant.

The composition can consist essentially of:
(a) 25-30% of an uncoated NSAID having an average particle size of less than 100 μm or a pharmaceutically acceptable salt thereof;
(b) 5-10% of a taste-masking agent;
(c) 1-10% of a disintegrant; and
(d) 2-25% of a binder.

The average particle size of the uncoated NSAID can be from 40-60 μm. A preferred average particle size of the uncoated NSAID is 50 μm. The composition can further include one or more of 40-50% filler, 0.5-5% lubricant and 0.1-5% glidant.

The composition can consist essentially of:
(a) 25-30% of an uncoated NSAID having an average particle size of less than 100 μm or a pharmaceutically acceptable salt thereof;
(b) 5-10% of a taste-masking agent;
(c) 1-10% of a disintegrant; and
(d) 5-15% of a binder.

The average particle size of the uncoated NSAID can be from 40-60 μm. A preferred average particle size of the uncoated NSAID is 50 μm. The composition can further include one or more of 40-50% filler, 0.5-5% lubricant and 0.1-5% glidant.

The composition can consist essentially of:
(a) 28-30% of an uncoated NSAID having an average particle size of less than 100 μm or a pharmaceutically acceptable salt thereof;
(b) 6-8% of a taste-masking agent;
(c) 3-5% of a disintegrant; and
(d) 8-12% of a binder.

The average particle size of the uncoated NSAID can be from 40-60 μm. A preferred average particle size of the uncoated NSAID is 50 μm. The composition can further include one or more of 42-47% filler, 0.75-1.5% lubricant and 0.5-1.5% glidant.

The composition can consist essentially of:
(a) 25-30% of uncoated ibuprofen having an average particle size of less than 100 μm or a pharmaceutically acceptable salt thereof;
(b) 5-15% of microcrystalline cellulose;
(c) 5-10% of malic acid; and
(d) 1-10% of crospovidone.

The average particle size of the uncoated ibuprofen can be from 40-60 μm. A preferred average particle size of the uncoated ibuprofen is 50 μm. The composition can further include one or more of 0.1-5% of colloidal silica, 40-50% of mannitol and 0.5-5% of sodium stearyl fumarate.

The composition can consist essentially of:
(a) 28-30% of uncoated ibuprofen having an average particle size of less than 100 μm or a pharmaceutically acceptable salt thereof;
(b) 8-12% of microcrystalline cellulose;
(c) 6-8% of malic acid; and
(d) 3-5% of crospovidone.

The average particle size of the uncoated ibuprofen can be from 40-60 μm. A preferred average particle size of the uncoated ibuprofen is 50 μm. The composition can further include one or more of 0.5-1.5% of colloidal silica, 42-47% of mannitol and 0.75-1.5% of sodium stearyl fumarate.

The composition can consist essentially of:
(a) 20-35% of an uncoated NSAID having an average particle size of less than 100 μm or a pharmaceutically acceptable salt thereof;
(b) 20-70% of a filler;
(c) 0.5-5% of a lubricant;
(d) 5-10% of a taste-masking agent;
(e) 1-10% of a disintegrant; and
(f) 2-25% of a binder.

The average particle size of the uncoated NSAID can be from 40-60 μm. A preferred average particle size of the uncoated NSAID is 50 μm. The composition can further include 0.1-5% of a glidant.

The composition can consist essentially of:
(a) 25-30% of an uncoated NSAID having an average particle size of less than 100 μm or a pharmaceutically acceptable salt thereof;
(b) 40-50% of a filler;
(c) 0.5-5% of a lubricant;
(d) 5-10% of a taste-masking agent;
(e) 1-10% of a disintegrant; and
(f) 2-25% of a binder.

The average particle size of the uncoated NSAID can be from 40-60 μm. A preferred average particle size of the uncoated NSAID is 50 μm. The composition can further include 0.1-5% of a glidant.

The composition can consist essentially of:
(a) 20-35% of an NSAID having an average particle size of less than 100 μm or a pharmaceutically acceptable salt thereof;
(b) 0.1-5% of a glidant;
(c) 20-70% of a filler;
(d) 0.5-5% of a lubricant;
(e) 2-25% of a binder;
(f) 5-10% of a taste-masking agent; and
(g) 1-10% of a disintegrant.

The average particle size of the uncoated NSAID can be from 40-60 μm. A preferred average particle size of the uncoated NSAID is 50 μm.

The composition can consist essentially of:
(a) 25-30% of an uncoated NSAID having an average particle size of less than 100 μm or a pharmaceutically acceptable salt thereof;
(b) 0.1-5% of a glidant;
(c) 40-50% of a filler;
(d) 0.5-5% of a lubricant;
(e) 5-15% of a binder;
(f) 5-10% of a taste-masking agent; and
(g) 1-10% of a disintegrant.

The average particle size of the uncoated NSAID can be from 40-60 μm. A preferred average particle size of the uncoated NSAID is 50 μm.

The composition can consist essentially of:
(a) 28-30% of an uncoated NSAID having an average particle size of less than 100 μm or a pharmaceutically acceptable salt thereof;
(b) 0.5-1.5% of a glidant;
(c) 42-47% of a filler;
(d) 0.75-1.5% of a lubricant;
(e) 8-12% of a binder;
(f) 6-8% of a taste-masking agent; and
(g) 3-5% of a disintegrant.

The average particle size of the uncoated NSAID can be from 40-60 μm. A preferred average particle size of the uncoated NSAID is 50 μm.

The composition can consist essentially of:
(a) 25-30% of uncoated ibuprofen having an average particle size of less than 100 μm or a pharmaceutically acceptable salt thereof;
(b) 0.1-5% of colloidal silica;
(c) 40-50% of mannitol;
(d) 0.5-5% of sodium stearyl fumarate;
(e) 5-15% of microcrystalline cellulose;
(f) 5-10% of malic acid; and
(g) 1-10% of crospovidone.

The average particle size of the uncoated ibuprofen can be from 40-60 μm. A preferred average particle size of the uncoated ibuprofen is 50 μm.

The composition can consist essentially of:
(a) 28-30% of uncoated ibuprofen having an average particle size of less than 100 μm or a pharmaceutically acceptable salt thereof;
(b) 0.5-1.5% of a colloidal silica;
(c) 42-47% of a mannitol;
(d) 0.75-1.5% of a sodium stearyl fumarate;
(e) 8-12% of a microcrystalline cellulose;
(f) 6-8% of a malic acid; and
(g) 3-5% of a crospovidone.

The average particle size of the uncoated ibuprofen can be from 40-60 μm. A preferred average particle size of the uncoated ibuprofen is 50 μm.

The composition of the present invention can have a friability of up to 2% w/w and a hardness of between 40N and 100N. The composition of the present invention can have a friability of less than 1% w/w and a hardness of greater than 50N. The composition of the present invention can have a friability of less than 1% w/w and a hardness of greater than 60N. The composition of the present invention can have a friability of less than 1% w/w and a hardness of greater than 70N. The composition of the present invention can have a friability of less than 1% w/w and a hardness of greater than 80N. The composition of the present invention can have a friability of less than 0.5% w/w and a hardness of greater than 50N. The composition of the present invention can have a friability of less than 0.5% w/w and a hardness of greater than 60N. The composition of the present invention can have a friability of less than 0.5% w/w and a hardness of greater than 70N. The composition of the present invention can have a friability of less than 0.5% w/w and a hardness of greater than 80N.

Preferably, the composition of the present invention can have a friability of less than 1% w/w and a hardness of greater than 80N. More preferably, the composition of the present invention can have a friability of less than 0.5% w/w and a hardness of greater than 80N.

Preferably, the composition can consist essentially of:
(a) 25-30% of an uncoated NSAID having an average particle size of 50 μm or a pharmaceutically acceptable salt thereof;
(b) 40-50% of a filler;
(c) 0.5-5% of a lubricant;
(d) 5-10% of a taste-masking agent;
(e) 1-10% of a disintegrant; and
(f) 2-25% of a binder.
   wherein the composition has a friability of less than 0.5% w/w and a hardness of greater than 80N.

More preferably, the composition can consist essentially of:
(a) 28-30% of an uncoated NSAID having an average particle size of 50 μm or a pharmaceutically acceptable salt thereof;
(b) 0.5-1.5% of a glidant;
(c) 42-47% of a filler;
(d) 0.75-1.5% of a lubricant;
(e) 8-12% of a binder;
(f) 6-8% of a taste-masking agent; and
(g) 3-5% of a disintegrant.
   wherein the composition has a friability of less than 0.5% w/w and a hardness of greater than 80N.

Most preferably, the composition can consist essentially of:
(a) 28-30% of uncoated ibuprofen having an average particle size of 50 μm or a pharmaceutically acceptable salt thereof;
(b) 0.5-1.5% of a colloidal silica;
(c) 42-47% of a mannitol;
(d) 0.75-1.5% of a sodium stearyl fumarate;
(e) 8-12% of a microcrystalline cellulose;
(f) 6-8% of a malic acid; and
(g) 3-5% of a crospovidone.
   wherein the composition has a friability of less than 0.5% w/w and a hardness of greater than 80N.

In a further aspect of the present invention there is provided the use of malic acid as a taste-masking agent in a composition that comprises uncoated ibuprofen having an average particle size of 40-60 μm. Preferably, the average particle size of the uncoated ibuprofen is 50 μm.

The ratio by weight of the uncoated ibuprofen and the malic acid in the composition can be from 1:1-8:1. A preferred ratio is 3:1-5:1. A most preferred ratio is 4:1 by weight.

Preferably, the present invention provides the use of malic acid as a taste-masking agent in a composition that comprises uncoated ibuprofen having an average particle size of 50 μm wherein the ratio by weight of the uncoated ibuprofen and the malic acid is 4:1.

For the avoidance of doubt the percentages expressed above are the percentage weight of the composition.

For the avoidance of doubt the term ‘uncoated’ refers to the ibuprofen prior to incorporation into the composition. The ibuprofen particles of the present invention are blended with the silica such that there is an admixture between the ibuprofen and silica rather than any surface modification of the ibuprofen by the silica.

For the avoidance of doubt the term ‘average particle size’ refers to the mean particle size of the ibuprofen used in the composition. For example, an average particle size of 50 μm means that the ibuprofen has a mean particle size of 50 μm.

The ibuprofen particles of the present invention are blended with the silica such that there is an admixture between the ibuprofen and silica rather than any surface modification of the ibuprofen by the silica.

For the avoidance of doubt the term ‘orodispersible tablet’ as used in the present specification refers to a solid single-dose preparation consisting of an uncoated tablet which is placed in the moth where it disperses rapidly in saliva before being swallowed.

Embodiments of the present invention will now be described, by way of example only, with reference to the accompanying FIGURES in which:

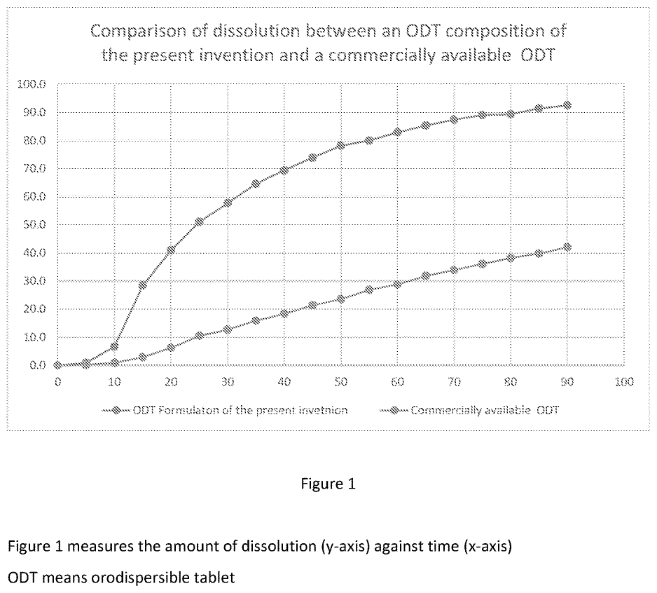

FIG. 1 illustrates dissolution data for compositions of both the present invention and the prior art.

### EXAMPLES

| Component | Example 1 (wt %) | Example 2 (wt %) |
|---|---|---|
| Ibuprofen | 28.57 | 28.57 |
| Colloidal Silica | 1.00 | 1.00 |
| Mannitol | 45.63 | 44.18 |
| Microcrystalline cellulose | 10.00 | 10.00 |
| Crospovidone | 4.00 | 4.00 |
| Malic Acid | 7.14 | 7.14 |
| Sodium stearyl fumarate | 1.25 | 1.25 |
| Sucralose | 0.86 | 0.86 |
| Flavour | 1.55 | 3.00 |
| TOTAL | 100.00 | 100.00 |

The orodispersible tablet may be prepared by a process involving dry blending or wet or dry granulation. However, it is preferred to use a manufacturing method which involves direct compression into an orodispersible tablet without an intermediate, e.g. a wet or dry granulation stage.

The composition may be made by dry mixing the active ingredient with one or more of the other ingredients, e.g. the lubricant and diluents and disintegrant, e.g. in a powder blending machine. The active agent can be blended with the silica. The remaining ingredients can be blended together separately. The two mixtures can be then be blended together. It is particularly preferred that the active ingredients are dispersed by progressive dilution with agitation in a proportion, e.g. about one-half, of the excipients so as to achieve even distribution of the active ingredient in the excipients, and then to add the remainder of the excipients with further agitation and mixing. The mixture may then be compressed in a tablet forming machine.

The taste and mouthfeel characteristics of the compositions were assessed. Participants in the assessment of the compositions of the present invention were provided with 2 tablets—the first was an orodispersible tablet in accordance with the present invention and the second was a sample of a commercially available orodispersible tablet.

The participants sampled each orodispersible tablet and recorded their observations for each characteristic by giving a rating out of 10. The participants were given water to cleanse their mouth between sampling each orodispersible tablet.

The table below illustrates the assessment average of 10 participants who compared the orodispersible tablets.

| | Example of present | Comparative |
|---|---|---|
| Characteristic | invention (Ave) | Example (Ave) |
| Acceptability of mouth sensation | 7.5 | 6.7 |
| Acceptability of texture | 6.8 | 5.6 |
| Acceptability of flavour | 7.7 | 6.7 |
| Acceptability of duration in mouth | 6.9 | 6.2 |
| Overall Rating | 7.2 | 6.6 |

As can be seen the composition of the present invention was given a significantly higher rating than the comparative example by the panellists.

FIG. 1 illustrates the dissolution profile for a composition of the present invention and an orodispersible tablet that is currently available. As can be seen, the dissolution of the ibuprofen in the orodispersible tablets of the present invention is significantly faster than that of a comparable existing tablet.

The dissolution of the ibuprofen was measured using a pH change dissolution method. The pH change method uses standard Ph Eur paddle apparatus and UV detection. This method is designed to more accurately reflect the change in pH that occurs between the stomach and the intestine and provide a more bio-relevant comparison for products. A composition of the present invention and a current commercially available orodispersible tablet are subjected to 9.5 minutes dissolution in 500 ml of pH 1.2 (using 0.1M HCL) and then to this is added 400 ml of a concentrated buffer solution. The dissolution is continued until the defined endpoint (this final solution has a pH of between 6-8). The amount of ibuprofen dissolved is measured via UV absorbance every 5 minutes up to 90 minutes.

Hardness and friability were measured using the relevant European Pharmacopoeial methods as outlined in sections 2.9.8 and 2.9.7 respectively.

An advantage of the present invention is that there is provided an orodispersible tablet that comprises an uncoated ibuprofen particle and has a good organoleptic profile with effective taste-masking, disintegrates in the mouth in less than one minute. As can be seen from FIG. 1, the use of an uncoated ibuprofen particle results in a significantly better dissolution profile than the dissolution profile of a product that comprises a coated ibuprofen.

The present invention is not intended to be limited to the exemplary embodiments described herein. Further modifications can be made without departing from the scope of the invention described herein.

1. A composition in the form of an orodispersible tablet comprising a non-steroidal anti-inflammatory drug (NSAID) and a taste-masking agent wherein the NSAID is uncoated and has an average particle size of less than 100 μm.

2. The composition as claimed in claim 1, wherein the average particle size of the uncoated NSAID is 40-60 μm.

3. The composition as claimed in claim 2, wherein the average particle size is 50 μm.

4. The composition as claimed in claim 1, wherein the NSAID comprises ibuprofen and its pharmaceutically acceptable salts or hydrates.

5. The composition as claimed in claim 1, wherein the taste-masking agent is selected from the group consisting of ascorbic acid, monosodium citrate, fumaric acid, malic acid, and combinations thereof.

6. The composition as claimed in claim 5, wherein the taste-masking agent is malic acid.

7. The composition as claimed in claim 5, wherein the amount of taste-masking agent is 0.1-15% by weight of the composition.

8. The composition as claimed in claim 7, wherein the amount of malic acid is 5-10% by weight.

9. The composition as claimed in claim 7, wherein the amount of malic acid is 7-9% by weight.

10. The composition as claimed in claim 1, wherein the ratio by weight of the non-steroidal anti-inflammatory drug (NSAID) and the taste-masking agent in the composition can be from 1:1-8:1.

11. The composition as claimed in claim 10, wherein the ratio of the NSAID and the tastemasking agent is 3:1-5:1.

12. The composition as claimed in claim 11, wherein the ratio of the NSAID and the tastemasking agent is 4:1 by weight.

13. The composition as claimed in claim 1, wherein the composition comprises a blend of a cellulosic binder, a sugar alcohol filler and a disintegrant.

14. The composition as claimed in claim 13, wherein the cellulosic binder is microcrystalline cellulose, the sugar alcohol is mannitol and the disintegrant is cross-linked polyvinylpyrrolidone (crospovidone).

15. The composition as claimed in claim 13, wherein the composition contains 2-4 parts by weight of cellulosic binder per part by weight of disintegrant, and 8-12 parts by weight of sugar alcohol binder per part by weight of disintegrant.

16. The composition as claimed in claim 15, wherein the composition contains cellulosic binder, sugar alcohol filler and disintegrant in a weight ratio of about of 3:11-12:1.

17. The composition as claimed in claim 1, wherein the composition has a friability of up to 2% w/w and a hardness of between 40N and 100N.

18. The composition as claimed in claim 17, wherein the composition has a friability of less than 1% w/w and a hardness of greater than 50N.

19. The composition as claimed in claim 17, wherein the composition has a friability of less than 0.5% w/w and a hardness of greater than 80N.

20. The composition as claimed in claim 1, wherein the composition comprises:
an uncoated non-steroidal anti-inflammatory drug (NSAID);
a taste-masking agent; and
a disintegrant,
wherein the uncoated NSAID has an average particle size of less than 100 μm, the taste-masking agent is malic acid which is present in an amount of 0.1-15% by weight of the composition, and the disintegrant is cross-linked polyvinylpyrrolidone (crospovidone) which is present in an amount of 1-10% by weight of the composition.

21. The composition as claimed in claim 1, wherein the composition comprises:
an uncoated non-steroidal anti-inflammatory drug (NSAID);
a taste-masking agent; and
a disintegrant,
wherein the uncoated NSAID has an average particle size of less than 100 μm, the taste-masking agent is malic acid which is present in an amount of 0.1-15% by weight of the composition, and
wherein the ratio of the uncoated non-steroidal anti-inflammatory drug (NSAID) to taste-masking agent is 4:1 by weight.

22. The composition as claimed in claim 19, wherein the composition further comprises a disintegrant which is cross-linked polyvinylpyrrolidone (crospovidone) and which is present in an amount of 1-10% by weight of the composition.

23. The composition as claimed in claim 1, wherein the composition comprises ibuprofen, a taste-masking agent and a disintegrant wherein the weight ratio of ibuprofen:taste-masking agent:disintegrant is from about 10:5:1 to about 5:1:1.

24. The composition as claimed in claim 23 wherein the weight ratio of ibuprofen:taste-masking agent:disintegrant is from about 8:2:1 to about 7:1:1.

25. The composition as claimed in claim 1, wherein the composition comprises:
uncoated ibuprofen;
a taste-masking agent; and
a disintegrant,
wherein the uncoated ibuprofen has an average particle size of 50 μm, the taste-masking agent is malic acid which is present in an amount of 7-9% by weight of the composition,
wherein the ratio of the uncoated ibuprofen to taste-masking agent is 4:1 by weight and the disintegrant is cross-linked polyvinylpyrrolidone (crospovidone) which is present in an amount of 3-6% by weight of the composition, and
wherein the weight ratio of ibuprofen:taste-masking agent:disintegrant is from about 8:2:1 to about 7:1:1.

26. The composition as claimed in claim 1, wherein the composition comprises:
an uncoated non-steroidal anti-inflammatory drug (NSAID);
a taste-masking agent;
a binder;
a disintegrant; and
a filler,
wherein the uncoated NSAID has an average particle size of less than 100 μm, the taste-masking agent is present in an amount of 0.1-15% by weight of the composition,
wherein the ratio of the NSAID to taste-masking agent is 4:1 by weight, and
wherein the composition contains 2-4 parts by weight of binder per part by weight of disintegrant, and 8-12 parts by weight of filler per part by weight of disintegrant.

27. The composition as claimed in claim 26, wherein the composition is in the form of an orodispersible tablet comprising:
uncoated ibuprofen,
a taste-masking agent,
a cellulosic binder,
a disintegrant and
a sugar alcohol filler,
wherein the ibuprofen has an average particle size of 50 μm, the taste-masking agent is malic acid and is present in an amount of 0.1-15% by weight of the composition,
wherein the ratio of the uncoated ibuprofen to taste-masking agent is 4:1 by weight, the cellulosic binder is microcrystalline cellulose and is present in an amount of 2-25% by weight of the composition, the disintegrant is cross-linked polyvinylpyrrolidone (crospovidone) and is present in an amount of 1-10% by weight of the composition, the filler is mannitol and is present in an amount of 20-70% by weight of the composition,
wherein the composition contains 2-4 parts by weight of cellulosic binder per part by weight of disintegrant, and 8-12 parts by weight of sugar alcohol filler per part by weight of disintegrant, and
wherein the weight ratio of ibuprofen:malic acid:cross-linked polyvinylpyrrolidone is from about 10:5:1 to about 5:1:1.

28. The composition as claimed in claim 1, wherein the composition comprises:
20-35% of an uncoated NSAID having an average particle size of less than 100 μm or a pharmaceutically acceptable salt thereof;
20-70% of a filler;
0.5-5% of a lubricant;
5-10% of a taste-masking agent; and
1-10% of a disintegrant.

29. The composition as claimed in claim 28 wherein the composition comprises:
25-30% of an uncoated NSAID having an average particle size of less than 100 μm or a pharmaceutically acceptable salt thereof;
40-50% of a filler;
0.5-5% of a lubricant;
5-10% of a taste-masking agent; and
1-10% of a disintegrant.

30. The composition as claimed in claim 28, wherein the composition further comprises one or more additional excipients selected from:
0.1-5% of a glidant; and
2-25% of a binder.

31. The composition as claimed in claim 28, wherein the composition further comprises:
28-30% of an uncoated NSAID having an average particle size of less than 100 μm or a pharmaceutically acceptable salt thereof;
0.5-1.5% of a glidant;
42-47% of a filler;
0.75-1.5% of a lubricant;
8-12% of a binder;
6-8% of a taste-masking agent; and
3-5% of a disintegrant.

32. The composition as claimed in claim 28, wherein the composition comprises:
25-30% of uncoated ibuprofen having an average particle size of less than 100 μm or a pharmaceutically acceptable salt thereof;
0.1-5% of colloidal silica;
40-50% of mannitol;
0.5-5% of sodium stearyl fumarate;
5-15% of microcrystalline cellulose;
5-10% of malic acid; and
1-10% of crospovidone.

33. The composition as claimed in claim 28, wherein the composition comprises:
28-30% of uncoated ibuprofen having an average particle size of less than 100 μm or a pharmaceutically acceptable salt thereof;
0.5-1.5% of colloidal silica;
42-47% of mannitol;
0.75-1.5% of sodium stearyl fumarate;
8-12% of microcrystalline cellulose;
6-8% of malic acid; and
3-5% of crospovidone.

34. The composition as claimed in claim 28, wherein the average particle size of the uncoated ibuprofen is from 40-60 μm.

35. A method of using malic acid as a taste-masking agent in a composition in the form of an orodispersible tablet that comprises an uncoated ibuprofen particle having an average particle size of 40-60 μm.

36. The method of use as claimed in claim 35, wherein the average particle size of the uncoated ibuprofen is 50 μm.

37. The method of use as claimed in claim 35, wherein the ratio by weight of the uncoated ibuprofen particle and the malic acid in the composition is from 1:1-8:1.

38. The method of use as claimed in claim 37, wherein the ratio by weight of the uncoated ibuprofen particle and the malic acid in the composition is from 3:1-5:1.

39. The method of use as claimed in claim 38, wherein the ratio by weight of the uncoated ibuprofen particle and the malic acid in the composition is from 4:1.

40. A method of using malic acid as a taste-masking agent in a composition in the form of an orodispersible tablet that comprises an uncoated ibuprofen particle having an average particle size of 50 μm wherein the ratio by weight of the uncoated ibuprofen particle and the malic acid is 4:1.
